(19) United States
(12) Patent Application Publication
(10) Pub. No.: US 2007/0264652 A1
(43) Pub. Date: Nov. 15, 2007

(54) Method for detection of a sequence

(75) Inventors: Pramod Upadhyay, New Delhi (IN); Sangeeta Bhaskar, New Delhi (IN)

Correspondence Address: OLIFF & BERRIDGE, PLC, P.O. BOX 19928, ALEXANDRIA, VA, 22320, US

(73) Assignee: NATIONAL INSTITUTE OF IMMUNOLOGY, NEW DELHI (IN)

(21) Appl. No.: 11/714,097

(22) Filed: Mar. 6, 2007

(30) Foreign Application Priority Data
May 10, 2006 (IN) 1202/DEL/2005

(51) Int. Cl. C12Q 1/68

(52) U.S. Cl. 435/6

(57) ABSTRACT

The present invention provides a novel method for detection of a sequence in a given sample, comprising the steps of isolating the target sequence from the given sample, providing a first and second probe comprising an oligo-nucleotide sequences complementary to respective ends of the target sequences and conjugated to a micro particle, contacting the probe with the target sequence under conditions to allow hybridization of the probes with the target sequence, exposing the target sequence to a reducing agent until appearance of a distinctive colour.

### FIELD OF THE INVENTION

The present invention relates to a novel method for detection of a sequence in a sample. The invention also provides a kit for detection of a sequence in a sample.

### BACKGROUND OF THE INVENTION

Sequence-selective DNA detection has become increasingly important for the genetic based diagnosis of disease. In last few years, some strategies for DNA detection based on functionalized colloidal gold have been reported. Nanoparticle-based detection schemes using two gold particle probes with covalently bound oligonucleotides complementary to a target of interest have also been reported [re: Elghani, R., J. J. Storhoff, R. C. Mucic, R. L. Letsinger and C. A. Mirkin. 1997. Selective Colorimetric Detection of Polynucleotides Based on the Distance-Dependent Optical Properties of Gold Nanoparticles. Science. 277:1078-1081 and Reynolds, R. A., C. A. Mirkin and R. L. Letsinger. 2000. A gold nanoparticle/latex microsphere-based colorimetric oligonucleotide detection method. Pure Appl. Chem. 72: 229-235]. When encountering target strands, these particle probes are polymerized and form network structures accompanied by a red-to-blue color change. By incorporating the sophistication of chip technology, better detection limits and convenience have been achieved in some of the reports. These include an array based electrical detection of DNA, a Raman spectroscopy based detection, a magnetic separation followed by chip based detection and a novel optical detection system. Gold colloid probe has also been used to detect single point mutation by quenching fluorescence. Zeptomole quantities of nucleic acid has been detected by a colorimetric scatter-based method [re: Storhoff, J. J., A. D. Lucas, V. Garimella, Y. P. Bao and U. R. Muller. 2004. Homogeneous detection of unamplified genomic DNA sequences based on calorimetric scatter of gold nanoparticle probes. Nat. Biotechnol. 22:883-887]. All these methods have different levels of sophistication and in most of these reports only the proof of principle is demonstrated. Methods in which final results are read by spectroscopy or chip based techniques may not be suitable for field level laboratories. Method with ultra low detection limits will require very high level of cleanliness to avoid cross contaminations. Otherwise simple to carry out un-conjugated gold colloid method will require very stringent control over the ionic strength of test samples which may not be easy when DNA is extracted from clinical samples.

Thus, there is a need in the art to provide a method for detection of desired sequence in a given sample such that the method may be employed even under harsh field conditions and which does not require stringent control over the sample analysed.

### DESCRIPTION OF THE INVENTION

Accordingly, the invention provides a novel method for detection of a sequence in a given sample, comprising the steps of:
a) isolating the target sequence from the given sample, the target having a first end and a second end;
b) providing a first probe comprising an oligo-nucleotide sequence complementary to a first end of the target sequence and conjugated to a first micro particle;
c) providing a second probe comprising an oligo-nucleotide sequence complementary to a second end of the target sequence and conjugated to a second micro particle;
d) contacting the first probe with a first end of the target sequence and the second probe with the second end of the target sequence under conditions to allow hybridization of the probes with the target sequence;
e) optionally washing away unbound probes;
f) exposing the target sequence of step (d) to a reducing agent which is added until a distinctive colour visible to the naked eye is detected, the appearance of the colour being indicative of the presence of the target sequence.

In one aspect, the method of the invention may be applied for detection of any specific sequence. The first probe in the method may be conjugated with colloidal gold as a micro particle and the second probe may be conjugated with latex bead.

Thus, the invention provides a detection method which is very simple to perform, does not involve any instrumentation to view final results and has been evaluated on relevant clinical samples. It can be completed in a few minutes.

The invention also provides a kit for detection of a sequence comprising a first and second probe, a reducing agent and manual of instructions.

To describe in detail, two nucleotide probes were synthesized corresponding to both ends of the target strand. One of the nucleotide sequences was covalently bound to latex beads and the other nucleotide was linked to the colloidal gold particles. Both of these probes get bound to respective ends of the target strand in a reaction mix. When filtered, the bound and unbound latex beads are retained on the filter. Bound latex beads have gold colloid particles attached at the other end of the target strand. This captured DNA having gold colloid attached at one of the ends is almost ‘invisible’ on the filter. Direct visual detection of target DNA is made possible by autometallography or silver enhancement. In this process the DNA captured by functionalized colloidal gold and latex beads is exposed to silver ions containing a reducing agent such as hydroquinone. Gold colloid particles provide nucleation for the deposition of reduced silver from silver ions and get enlarged to between 30 nm and 100 nm in diameter and become distinctive black in color. The detection scheme is shown in FIG. 1. While the detection scheme may be applied to any biological material such as nucleotide sequence, antibodies, peptides etc., the following is an example performed to detect the IS6110 gene of M. tuberculosis.

### DESCRIPTION OF ACCOMPANYING DRAWINGS

FIG. 1 is a diagrammatic representation depicting the detection scheme of the invention using Gold colloid—latex bead test. The reaction mix contains target nucleotide (3) labelled with gold colloid (gold probe) (1) and nucleotide labelled with latex beads (latex probe) (2). If these nucleotides find the target strand, they get bound at respective ends (a). When this reaction mix is filtered and washed through a 0.22 micron membrane, bound and free latex probes are retained on the membrane and unbound gold probe is filtered out (b). During silver enhancement invisible gold colloid particles become sites for the nucleation and turn black due to the precipitation of a lot of silver metal (4) (c).

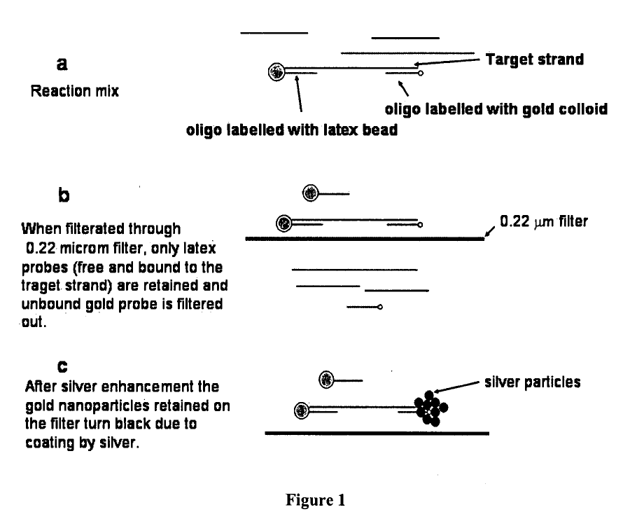

FIG. 2 is a diagrammatic representation showing the sequence of target strand, nucleotide labelled with latex beads and nucleotide labelled with gold colloid (a). After a single mutation in the target strand the nucleotide labelled with latex beads does not bind to it (b).

FIG. 3 (a) to (c) are photographs of filters showing the spots obtained with blank, 25 pg, 250 pg and 2.5 ng of target nucleotide after washing (top row) and after silver enhancement (bottom row). The spots are almost invisible after washing but become visible after silver enhancement (a). The variation of the colour intensity of the spots with the amount of target DNA is shown in panel (b). The response is not linear. Panel (c) shows a comparison of colour intensity of spots obtained from several types of DNA mixtures. The sensitivity of the test remains unaffected in a mixture of similar DNA or large amount of irrelevant DNA.

FIG. 4 is a boolean diagram showing summary of results obtained by different methods. It is important to note that all PCR positive samples were also Gold colloid—latex bead positive.

### EXAMPLES

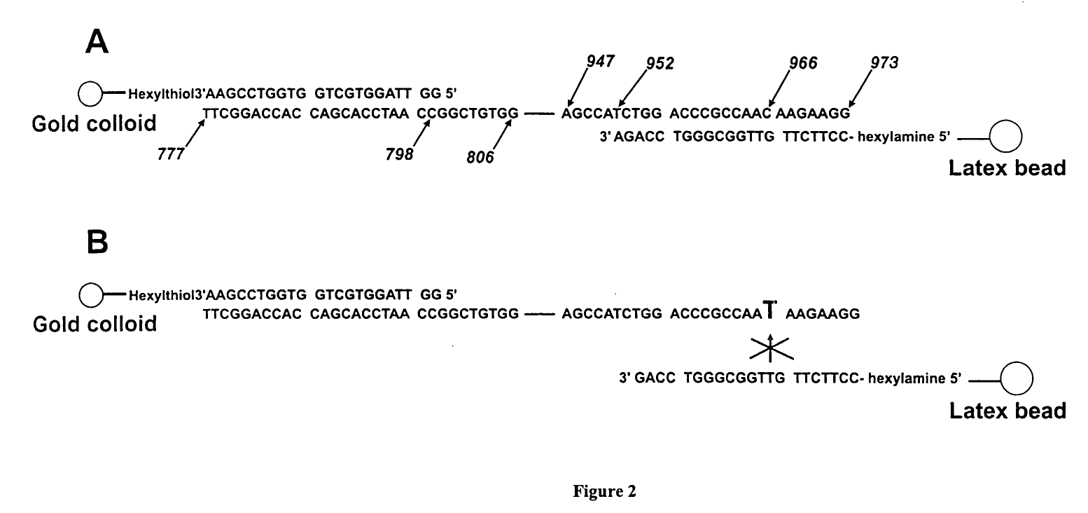

The potential of this gene for the diagnosis of tuberculosis has been demonstrated by a number of authors (17, 2, 10, 1, 6). The minimum detection limit of this test is 25 pg of synthetic nucleotide and 50 ng of whole cell DNA. The test is sensitive to detect single point mutation. The exact sequences of both the nucleotide probes, the target strand and ‘mutated strand’ is shown in FIG. 2.

This test was used to check the target gene in sputum samples and compared the results obtained with polymerase chain reaction (PCR). In contrast to PCR, in this detection method it is not necessary that the two target sequences are separated by a few bases. It is possible to design appropriate probes to target any unique sequence of 40 or more bases. Hence highly specific probes can be made with relative ease. The test does not require elaborate DNA extraction method. It is most suited for field level laboratories.

The experiments below are conducted first by using a synthetic target strand. Very high specificity of this technique has been demonstrated by a synthetic target strand in which one base was altered. The sensitivity of the test was estimated for the synthetic target stand and for the whole genomic DNA of M. tuberculosis. Finally, the test has been carried out on DNA extracted from sputum samples and results compared with PCR.

Materials Used

Preparation of colloidal gold

Gold chloride, HAuCl₄ (Sigma) was used. Colloidal gold with an average diameter of 25-30 nm was prepared by controlled reduction of a 0.02% boiling solution of gold chloride with 1% sodium citrate solution (3).

Target Sequence

The target sequence is from nucleotide number 776 to 972 of IS6110 element (18), GenBank accession no. M29899. For evaluating this assay, a nucleotide sequence shown in FIG. 2 was synthesized in which nucleotide number 807 to 945 were removed. The target strand was shortened to make its synthesis economical.

Sequence for conjugation to gold colloid and latex beads

3′ hexylthiol modified sequence was synthesized based on target sequence from nucleotide 776 to 754, similarly 5′ hexylamine modified sequence was synthesized from nucleotide 951 to 972. Both of these sequences are shown in FIG. 2.

Mutated sequence

In order to evaluate the ability of this test to differentiate single point mutation, a target sequence was synthesized in which at position 965 base, cytosine was replaced with thymine. In order to examine the specificity of the test, detection of the target strand was carried out in a large background of this ‘mutated’ strand.

Primers for PCR

Same nucleotide targets were used to make primers for the PCR. One of the primers was similar to hexylamine modified nucleotide and the other was complimentry to hexylthiol modified nucleotide sequence, but without hexylamine or hexylthiol modifications.

Conjugation of oligonucleotides to colloidal gold and latex beads

200 μl of above mentioned colloidal gold solution was mixed with 10 μl (around 25 ng) of hexylthiol modified nucleotide. This mix was incubated over night at room temperature. Gold bound nucleotide was washed twice with 1 ml of milliQ water and finally dispensed in 200 μl of milliQ water.

Carboxylate modified latex beads 0.45 μm (sigma) were used for conjugation to hexylamine modified nucleotide. 100 μl of beads were washed twice with milliQ water. Washed beads were dispersed in 1 ml PBS (10 mM) containing 0.1 mg 1-cyclohexyl-3-(2-morphoilino-ethyl)carbodimide metho-p-toluenesulfonate (sigma) and mixed with 50 μl (around 50 ng) hexylamine modified nucleotide and incubated over night at room temperature. Latex beads bound nucleotide were washed with milliQ water and finally dispersed in 1 ml milliQ water.

Silver enhancement reagents

For silver enhancement of almost invisible gold nanoparticles, equal volumes of 0.5% hydroquinone and 0.2% silver acetate were taken in 0.5 M citrate buffer (pH 3.8).

Preparation of Genomic DNA

Genomic DNA from M. tuberculosis culture was extracted by ethanol precipitation method. Briefly, 1% W/V glycine was added a day before harvesting. The culture was pelleted down and resuspended in lysis solution containing 10% sucrose and 2 mg/ml lysozyme in 50 mM Tris buffer. Proteinase K and SDS were then added and the mixture incubated at 55° C. Lysate was extracted with phenol saturated Tris EDTA and nucleic acid was precipitated with 100% ethanol, dissolved in TE buffer and estimated by measuring the absorbance at 260 nm and 280 nm.

Sputum samples

Sputum samples of suspected tuberculosis patients were collected and processed at New Delhi Tuberculosis Centre, New Delhi. These sputum samples were examined initially by microscopy and cultured by Petroff's method.

DNA extraction from sputum

DNA from sputum samples was extracted (method A) by NALC and GITC method (4). Sputum samples were mixed with 0.2 vol. of 2.5% N-acetyl L-cysteine (NALC) in 68 mM phosphate buffer, (pH 6.8) and allowed to stand for 10 mins, centrifuged at 25000 g. Pellet was re-suspended in inhibitor removal solution containing 5 M GITC, 25 mM EDTA, 0.5% sarcosyl, 0.2M βmercaptoethanol in 50 mM Tris-chloride, pH 7.5. After 30 min, the mixture was centrifuged at high speed and the pellet was washed and dried. DNA was extracted from this dried pellet by adding 5 vols of 10% suspension of Chelex 100 (Bio-Rad) containing 0.3% Tween 20 and 0.03% Triton X-100. Tubes were heated at 90° C. for 30 mins and supernatant was used for PCR and Gold colloid—latex bead test.

For Gold colloid—latex bead test, DNA was extracted (method B) by mixing equal volumes of sputum and 2.5% N-acetyl L-cysteine (NALC) in 68 mM phosphate buffer for 15 mins, centrifuged at high speed and pellet was washed and re-suspended in 1 ml of 10 mM TE buffer and placed over boiling water for 30 mins. The supernatant was used for Gold colloid—latex bead test.

PCR test

Qiagen Taq PCR core kit was used to carry out PCR. The reaction was started with a ‘hot start’ for 4 mins and 30 cycles of melting at 94° C. for 1 min, annealing at 64.5° C. for 45 secs, extension at 72° C. for 45 secs were carried out. The amplification was checked on ethidium bromide stained agrose gel.

Gold colloid—latex bead test procedure

In order to make single-stranded (ss) DNA, 10 μl of DNA sample extracted by any of the methods given above was kept in boiling water for 20 secs for melting of DNA and then in the freezing mixture for 20 secs for rapid cooling to prevent annealing. This heating cooling cycle was repeated three times. To this denatured DNA sample, 20 μl of latex beads probe, 10 μl of gold probe and 5 μl of 100 mM PBS were added. After 5 mins, the reaction mix was filtered through 0.22 micron PVDF hydrophilic filter (Millipore). Latex beads retained on the filter were washed with 10 ml milliQ water to remove unbound gold probe and chloride ions. Following this 50 μl silver enhancement solution was added on the latex beads. Filter was examined by naked eye after 30 mins for the black spot.

Results

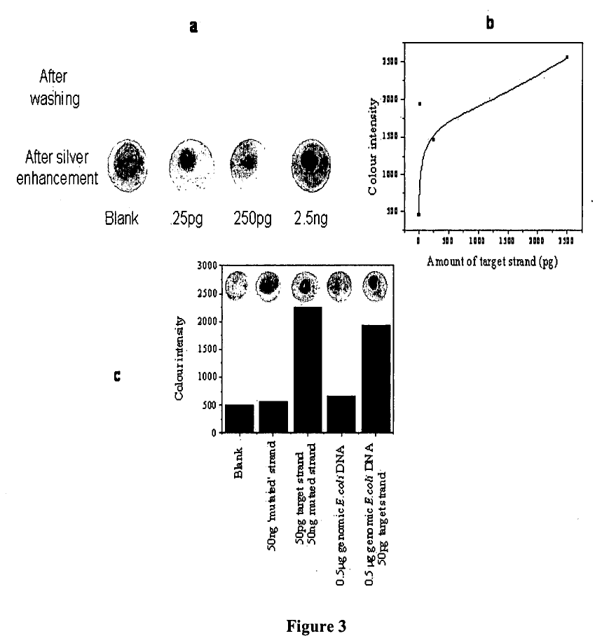

The lower detection limit of this test was estimated by performing it over a series of dilutions containing 0 to 2.5 ng of the target strand. These solutions of target strand were mixed with both the probes and the reaction mix were filtered and washed. FIG. 3a shows the photographs of these filters after silver enhancement. It is possible to identify the black spots indicating the presence of the target strand. Further, one can clearly distinguish the black spot corresponding to 25 pg DNA and the blank. The colour intensity of these spots was converted into a gray scale and measured by ImageJ software (http://rsb.info.nih.gov/ij/index.html) and a plot is shown in FIG. 3b. The colour intensity increase with amount of target DNA but the response is not linear and therefore the quantitative estimation of the target strand is difficult by this method.

To verify the applicability of this test for the detection of the target sequence in a mixture of similar or a lot of irrelevant DNA, a comparison of colour intensity obtained from several types of mixtures was performed. FIG. 3c shows the colour intensity obtained from blank, 50 ng of a ‘mutated’ strand, 50 pg of target strand+50 ng mutated strand, approximately 0.5 μg genomic DNA from E. coli. and 50 pg of target strand+0.5 μg genomic DNA from E. coli. The clear black spots obtained from the target strand whether it is present in a ‘mutated’ sequence or in a lot of junk DNA can be seen in FIG. 3C. These results show that it is possible to adapt this test to detect single point mutation and it remains sensitive to detect the target sequence in a large pool of nucleotides.

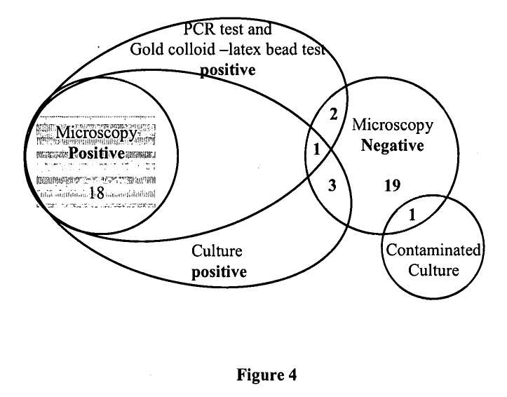

To establish the utility of this test as a diagnostic tool, it was carried out on DNA extracted from M. tuberculosis and the lower detection limit was found to be 50 ng with careful silver enhancement. Next, the test was performed on DNA extracted from sputum samples of suspected tuberculosis patients. The results are summarized in FIG. 4.

Results of conventional tests: 44 sputum samples were taken from patients attending a Tuberculosis clinic for the first time. Samples were analyzed the same day by microscopic examination and 18 out of 44 were found positive and treatment was started for positive patients. All the 18 microscopy positive samples were found positive by culture, Gold colloid—latex bead test and PCR.

Out of remaining 26 microscopy negative samples, 4 were found to be culture positive and 3 samples were Gold colloid—latex bead test positive. One sample was found to be positive by all the three tests, culture, Gold colloid—latex bead test and PCR. One of the cultures got contaminated.

In this study, it is of prime importance to compare the outcome of Gold colloid—latex bead test and PCR as both of these tests recognize the same gene. Out of the 44 samples, 21 were found to be Gold colloid—latex bead test positive and same samples were also PCR positive. The discrepancy of these data with culture results could be due to the limitation of tuberculosis diagnosis by IS6110 gene.

An evaluation of two DNA extraction procedures was also carried out. Identical results were obtained by Gold colloid—latex bead test with DNA extracted by either of DNA extraction methods, the Chelex 100 (method A) or boiling method (method B).

These results demonstrate that the sensitivity of Gold colloid—latex bead test is similar to PCR based test. A simplified DNA extraction method is likely to work for Gold colloid—latex bead test, as no amplification of a gene is done and inhibitors have a very limited role to play. However, as compared to PCR, this test is simple to carry out and results are visible through naked eyes. This test should find application in field level laboratories. For this purpose, a kit would be provided comprising a first and second probe, a reducing agent and manual of instructions.

We claim:

1. A novel method for detection of a sequence in a given sample, comprising the steps of:
a) isolating the target sequence from the given sample, the target having a first end and a second end;
b) providing a first probe comprising an oligo-nucleotide sequence complementary to a first end of the target sequence and conjugated to a first micro particle;
c) providing a second probe comprising an oligo-nucleotide sequence complementary to a second end of the target sequence and conjugated to a second micro particle;
d) contacting the first probe with a first end of the target sequence and the second probe with the second end of the target sequence under conditions to allow hybridization of the probes with the target sequence;
e) optionally washing away unbound probes;
f) exposing the target sequence of step (d) to a reducing agent which is added until a distinctive colour visible to the naked eye is detected, the appearance of the colour being indicative of the presence of the target sequence.

2. A method as claimed in claim 1, wherein the target sequence is a nucleotide sequence, DNA sequence, protein sequence, peptide sequence, antibody or a protein-nucleic acid sequence.

3. A method as claimed in claim 1, wherein the first and second micro-particle is selected from gold, silver, latex etc.

4. A method as claimed in claim 1, wherein the reducing agent is hydroquinone.

5. A kit for detection of a sequence comprising a first and second probe, a reducing agent and manual of instructions.


SEQUENCE LISTING (7 sequences)

SEQ ID NO: 1 - DNA, 1361 bp, Mycobacterium tuberculosis
cgatgaaccgccccggcatgtccggagactccagttcttggaaaggatggggtcatgtcaggtggttcatcgaggaggtacccgccggagctgcgtgagcgggcggtgcggatggtcgcagagatccgcggtcagcacgattcggagtgggcagcgatcagtgaggtcgcccgtctacttggtgttggctgcgcggagacggtgcgtaagtgggtgcgccaggcgcaggtcgatgccggcgcacggcccgggaccacgaccgaagaatccgctgagctgaagcgcttagcggcgggacaacgccgaattgcgaagggcgaacgcgattttaaagaccgcgtcggctttcttcgcggccgagctcgaccggccagcacgctaattaacggttcatcgccgatcatcagggccaccgcgagggccccgatggtttgcggtggggtgtcgagtcgatctgcacacagctgaccgagctgggtgtgccgatcgccccatcgacctactacgaccacatcaaccgggagcccagccgccgcgagctgcgcgatggcgaactcaaggagcacatcagccgcgtccacgccgccaactacggtgtttacggtgcccgcaaagtgtggctaaccctgaaccgtcagggcatcgaggtggccagatgcaccgtcgaacggctgatgaccaaactcggcctgtccgggaccacccgcggcaaagcccgcaggaccacgatcgctgatccggccacagcccgtcccgccgatctcgtccagcgccgcttcggaccaccagcacctaaccggctgtgggtagcagacctcacctatgtgtcgacctgggcagggttcgcctacgtggcctttgtcaccgacgcctacgctcgcaggatcctgggctggcgggtcgcttccacgatggccacctccatggtcctcgacgcgatcgagcaagccatctggacccgccaacaagaaggcgtactcgacctgaaagacgttatccaccatacggataggggatctcagtacacatcgatccggttcagcgagcggctcgccgaggcaggcatccaaccgtcggtcggagcggtcggaagctcctatgacaatgcactagccgagacgatcaacggcctatacaagaccgagctgatcaaacccggcaagccctggcggtccatcgaggatgtcgagttggccaccgcgcgctgggtcgactggttcaaccatcgccgcctctaccagtactgcggcgacgtcccgccggtcgaactcgaggctgcctactacgctcaacgccagagaccagccgccggctgaggtctcagatcagagagtctccggactcaccggggcggttcacga

SEQ ID NO: 2 - DNA, 197 bp, Mycobacterium tuberculosis
ttcggaccaccagcacctaaccggctgtgggtagcagacctcacctatgtgtcgacctgggcagggttcgcctacgtggcctttgtcaccgacgcctacgctcgcaggatcctgggctggcgggtcgcttccacgatggccacctccatggtcctcgacgcgatcgagcaagccatctggacccgccaacaagaagg

SEQ ID NO: 3 - DNA, 57 bp, Mycobacterium tuberculosis
ttcggaccaccagcacctaaccggctgtggagccatctggacccgccaacaagaagg

SEQ ID NO: 4 - DNA, 22 bp, Mycobacterium tuberculosis
ttcggaccaccagcacctaacc

SEQ ID NO: 5 - DNA, 22 bp, Mycobacterium tuberculosis
aagcctggtggtcgtggattgg

SEQ ID NO: 6 - DNA, 22 bp, Mycobacterium tuberculosis
tctggacccgccaacaagaagg

SEQ ID NO: 7 - DNA, 22 bp, Mycobacterium tuberculosis
agacctgggcggttgttcttcc